(19) United States
(12) Patent Application Publication
(10) Pub. No.: US 2025/0051267 A1
(43) Pub. Date: Feb. 13, 2025

(54) PROCESS FOR THE PREPARATION OF 2,4,6-TRIIODOISOPHTHALIC BISAMIDES

(71) Applicant: BRACCO IMAGING S.P.A., Milan (IT)

(72) Inventors: Luciano LATTUADA, Cassina De' Pecchi (IT); Luca BEVERINA, Milano (IT); Mauro SASSI, Vedano Olona (IT); Sara MATTIELLO, San Giuliano Milanese (IT); Antonio PAPAGNI, Milano (IT); Anita ZUCCHI, Carnate (IT)

(73) Assignee: BRACCO IMAGING S.P.A., Milan (IT)

(21) Appl. No.: 18/721,282

(22) PCT Filed: Dec. 15, 2022
(86) PCT No.: PCT/EP2022/086082

§ 371 Date: Jun. 18, 2024

(30) Foreign Application Priority Data
Dec. 20, 2021 (EP) 21215850.5

(51) Int. Cl. C07C 237/32

(52) U.S. Cl. CPC C07C 237/32

(57) ABSTRACT

The present invention relates to a novel process for the preparation of 2,4,6-triiodoisophthalic bisamide derivatives, which are useful intermediates in the preparation of non-ionic X-ray contrast agents, starting from the corresponding acyl chloride intermediates which are amidated in a mixture of specific hydrotropic solvents in the presence of a low amount of water. In addition, the invention relates to the use of such hydrotropic solvents in the preparation of said 2,4,6-triiodoisophthalic bisamide intermediates to produce non-ionic X-ray contrast agents.

The invention relates to a novel process for the preparation of 2,4,6-triiodoisophthalic bisamide derivatives, which are useful intermediates in the preparation of non-ionic X-ray contrast agents. More precisely, it relates to the amidation reaction of the corresponding acyl dichloride intermediates which is carried out in a mixture of specific hydrotropic solvents in the presence of a low amount of water. In addition, the invention relates to the use of such hydrotropic solvents in the preparation of said 2,4,6-triiodoisophthalic bisamide intermediates for the production of non-ionic X-ray contrast agents.

### BACKGROUND OF THE INVENTION

The invention relates to the preparation of intermediates useful for the synthesis of non-ionic X-ray contrast agents, including for instance iopamidol, iohexol, iopentol, iodixanol, ioversol, iomeprol and iobitridol (Lusic, H. et al, Chem. Rev. 2013, 113, 1641-1666), which are all characterized by the presence of a 2,4,6-triiodo-phenyl scaffold bearing two aminocarbonyl moieties in positions 1 and 3 (isophtalic acid derivatives).

The industrial processes currently used for the preparation of these compounds comprise the amidation reaction of the corresponding acyl chloride intermediates occurring during their synthesis. The formation of amide bonds, usually achieved by the reaction of acyl dichlorides with amines, is one of the most critical steps in the synthetic processes for the preparation of such compounds. In fact, such amidation process involves reacting water-soluble amines (forming the side chains) with water-insoluble substrates (comprising the triiodinated core), thus making difficult the selection of a suitable solvent for the reaction.

Typically, dipolar aprotic solvents, such as DMAC or DMF, are used in such transformations to overcome reactivity and/or solubility issues, since they are able to solubilize both lipophilic aryl intermediates and hydrophilic hydroxy-alkyl amines.

However, such solvents suffer from the disadvantage of being reprotoxic and flagged under the “Registration, Evaluation, Authorisation and restriction of Chemicals” (REACH) legislation as Substances of Very High Concern, especially when they are used in the bulk volumes of an industrial production. Other aprotic dipolar solvents, such as N-methylpyrrolidone (NMP) or N-ethylpyrrolidone (NEP), have been tested in these reactions with good results, but they are reprotoxic too, therefore not suitable to solve this safety issue. Moreover, they display a very high boiling point and they are difficult to be removed at the end of the reaction and from the bulk drug substance.

The regulators are more frequently inviting to the use of environmentally friendly solvent systems in the pharmaceutical industry. For instance, the use of less toxic solvents, such as alcohols, has been already described and proven to be efficient for the above transformations.

One approach towards the replacement of undesirable polar aprotic solvents relates for instance to the use of an aqueous micellar technology, such as that described by Shi M. et al., Org. Process Res. Dev 2020, 24:1543-1548. This article discloses the reaction of acyl chlorides with amines carried out in a 2 wt % aqueous solution of DL-alpha-tocopherol methoxypolyethylene glycol succinate (TPGS-750-M) in the presence of a base, like DIPEA. TPGS-750-M is a known surfactant endowed with amphiphilic nature and allows for reactions to proceed in a non-homogeneous system, e.g. in a suspension or emulsion, taking advantage of the micellar effect, the intrinsic solubility of the partners and the highly dynamic exchange between the bulk water and the lipophilic layer created by the micelle.

However, the micellar approach can present some limitations, such as for instance the difficulty of removing the surfactant at the end of the reaction, in particular with products highly soluble in water such as for instance Iopamidol and other non-ionic X-ray contrast agents.

Other documents disclose the use of different water-soluble solvents. For instance, patent EP1075462 discloses a process for the preparation of a 2,4,6-triiodo-1,3-benzenecarboxamide starting from the corresponding acyl dichloride, wherein the solvent DMAC, typically used in such reaction, is replaced by a solvent selected from a lower alcohol, monoalkyl ether glycols and cyclic, linear or branched alkyl ethers. For instance, in examples 6 and 7 a preparation of Iopamidol is described where 2-methoxyethanol or 3-ethoxyethanol solvents are used for the formation of the corresponding bisamide intermediate only in the presence of a very large excess of serinol, which is necessary to suppress the competitive esterification of the chloride with the solvent.

Patent application CN104098484 discloses that an amidation reaction (step c) of 2,4,6-triiodo-1,3-benzene-dicarboxylic acid or acyl chloride or carboxylate intermediates with protected serinol can be performed in alkaline conditions using a solvent selected from a lower alcohol, preferably tert-butanol and sec-butanol, a glycol monoalkyl ether, preferably 2-methoxyethanol, 2-ethoxyethanol, and a cyclic, linear or branched alkyl ether, preferably 1,4-dioxane, diglyme and methyl tert-butyl ether. However, no examples are therein provided of reactions with such solvents and all the exemplified amidations are still performed in the usual DMAC.

However, a drawback of such approaches is that even solvents like 2-methoxyethanol and 2-ethoxyethanol are characterized by a toxicity profile similar to that of DMAC, thus they cannot be considered a greener alternative and cannot solve the problem mentioned above.

As a consequence, there is a need for an improved process of bis-amidation of 2,4,6-triiodo-1,3-benzenedicarboxylic acid dichloride which uses a safer, economic and readily available alternative solvent and which could be applicable at industrial scale with good productivity results.

It has now been found that the above transformations can be easily and efficiently performed in a solvent selected from a sub-group of the family of hydrotropic solvents in the presence of low percentages of water.

Hydrotropes are a class of water-soluble compounds with an amphiphilic structure that can increase the solubility of hydrophobic substances in water by means other than micellar solubilization. Typically, hydrotropes consist of a hydrophilic part and a hydrophobic part (like surfactants), the latter however being generally too small to cause spontaneous self-aggregation and formation of micelles (Subbarao C. V. et al, Chem. Eng. Technol. 2012, 35:225-237; Dhapte V. et al, St. Petersburg Polytechnical University Journal: Physics and Mathematics 2015, 1:424-435).

A class of such compounds is represented for instance by the monoethers of alkylene glycols, which, based on the length of their alkyl chain, can behave as co-solvents, hydrotropes or real surfactants.

It has now surprisingly found that some hydrotropic ethers or glycols can act, in the presence of a low amount of water, as useful and safe solvents for carrying out the reaction of the invention, without the need to add any inorganic base. Moreover, the process of the invention, with the use of such group of ethers or glycols, allows to effectively overcome the mentioned problems related to the presence of DMAC, at the same time providing a final product with remarkable efficiency.

In fact, differently from the above mentioned synthetic procedures, the process of the present invention provides for 2,4,6-triiodoisophthalic bisamide intermediates with good yield results by using chemicals endowed with better safety features and more easily removable in order to minimize any involved risks of side-effects and toxic effects for the patients.

### SUMMARY OF THE INVENTION

In a first aspect, the aim of the present invention is to provide a process for the preparation of 2,4,6-triiodoisophthalic bisamide compounds by reacting the corresponding 2,4,6-triiodo-acyl chloride intermediates with a suitable aminoalcohol in the presence of hydrotropic ethers or glycols in the presence of a low amount of water. Such compounds are useful intermediates in the synthesis of non-ionic X-ray contrast agents and, according to the present invention, are prepared with a new method suitable for industrial scale production which is particularly safe and providing a selective high-yielding procedure.

Surprisingly, it has been found that such reaction can be conveniently performed in a mixture comprising a group of ethers or glycol derivatives with hydrotropic behavior in the presence of an amount of water ranging from 0.1% to 15% v/v.

In fact, being miscible with water in all proportions and having an amphiphilic structure, these specific solvents can increase the solubility of the hydrophobic substrates in an aqueous medium, without the processing issues of the micellar systems.

The amount of water ranging from 0.1% to 15% v/v has been found enough to help the solubilization of the reagents allowing to work with mild conditions, such as with temperatures close to ambient temperature, but also sufficiently low in order to avoid possible hydrolyzation issues and formation of undesired by-products. In some cases, the process of the invention can be easily carried out in the presence of a low amount of water such as 0.1% v/v or even in the absence of water. Amounts lower than 3% can be also represented by the water already present in the reagents' bottles, such as water of crystallization or hydration, so that it is not necessary to add water in the reactor as a separate solvent. In these cases, the hydrotropic ethers or glycols can be considered as solvating the reagents also without the further addition of water.

The above hydrotropic solvents are not reprotoxic, thus the use of a mixture of one of these solvents with a low percentage of water is extremely safer with respect to the use of dipolar aprotic solvents, like DMAC, for performing the reaction of the invention.

Moreover, these solvents display a relatively low boiling point and, especially, they are much more volatile than dipolar aprotic solvents and stable at the boiling point, so that they can be easily removed from the reaction mixture by direct evaporation (e.g. by distillation).

This advantageously makes the preparation process of the invention an easier, cheap and environmentally friendly method, which allows to replace harmful solvents and provides yields of at least 70% of the corresponding 2,4,6-triiodoisophthalic bisamide derivatives.

Moreover, the present invention further relates to the use of a mixture of such hydrotropic ethers and glycols in the presence of an amount of water ranging from 0.1% to 15% v/v in the preparation of 2,4,6-triiodoisophthalic bisamide derivatives useful in the synthesis of non-ionic X-ray contrast agents.

### DETAILED DESCRIPTION OF THE INVENTION

According to a first aspect, the present invention relates to a process for the preparation of a 2,4,6-triiodoisophthalic bisamide of formula (I)

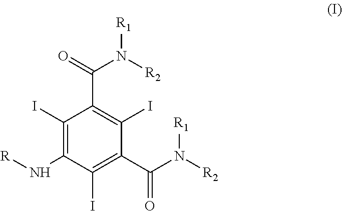

wherein
R is selected from hydrogen, linear or branched C₁-C₆ alkyl-carbonyl, optionally substituted by —OCOCH₃, and a group 2-(propan-2-yl)-1,3-dioxane-5-carbonyl;
R₁ is each independently hydrogen or methyl; and
R₂ is each independently a group —CH(CH₂OH)₂ or —CH₂CH(OH)CH₂OH, said process comprising the step of
a) reacting a substituted 2,4,6-triiodo-1,3-benzenedicarboxylic acid dichloride of formula (II)

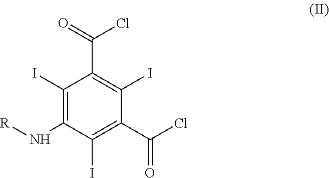

wherein R is as defined above,
with an amine of formula (III)

R₁—NH—R₂ (III)
wherein R₁ and R₂ are as defined above,
characterized in that the reaction of step a) is carried out in a mixture comprising a solvent selected from hydrotropic ethers and glycols in the presence of an amount of water ranging from 0.1% to 15% v/v and in that the molar ratio between the amine of formula (III) and the acyl chloride of formula (II) is comprised between 3:1 and 7:1.

In a preferred embodiment, said solvent in step a) is a compound of formula (IV)

R₃—O—R₄—OH (IV)
wherein
R₃ is a linear or branched C₁-C₆ alkyl and
R₄ is a linear or branched C₃-C₁₀ alkyl, optionally interrupted by at least one oxygen atom.

In another preferred embodiment of the invention, in the definition of the compounds of formula (I) R represents hydrogen or a group selected from the following:

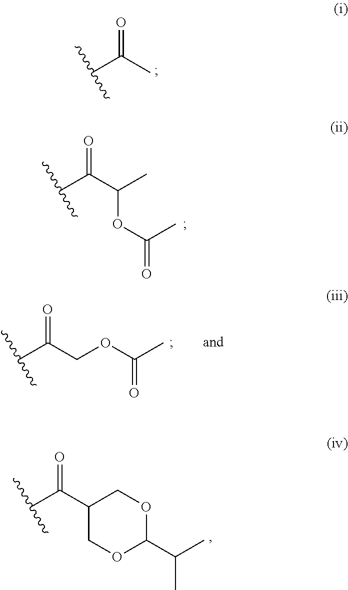

where [symbol] represents the bonding position.

In a more preferred embodiment, in the definition of the compounds of formula (I) R represents a group CH₃CO— (i), as defined above.

In another embodiment of the invention, the amine of formula (III) is an aminoalcohol selected from 2-amino-1,3-propanediol, 3-amino-1,2-propanediol and 3-(methylamino) propane-1,2-diol.

According to another embodiment, the present invention relates to the process as defined above further comprising the following steps:
b) hydrolyzing the compound of formula (I) and/or removing any protecting group to obtain the corresponding compound of formula (I′)

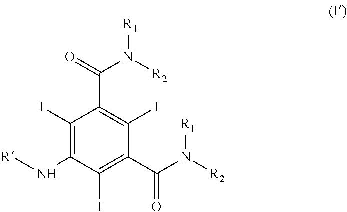

wherein R₁ and R₂ are as defined above and R′ is selected from a linear or branched C₁-C₆ alkyl-carbonyl, optionally substituted by one or more groups-OH; and/or
c) alkylating or amidating the compound of formula (I) or (I′) to obtain a compound of formula (I″)

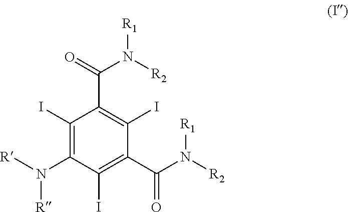

wherein R₁, R₂ and R′ are as defined above and R″ is a linear or branched C₁-C₆ alkyl or C₁-C₆ alkyl-carbonyl, optionally substituted by one or more groups-OH and/or —OCH₃.

In another embodiment, two equivalents of a compound of formula (I) can be reacted together with an alkylating agent in order to obtain a dimeric compound of formula (I′″)

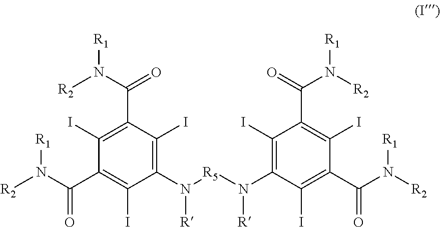

wherein R₁, R₂ and R′ are as defined above and R₅ is a linear or branched C₁-C₆ alkylene group, optionally substituted by one or more groups-OH.

If necessary, the compounds of formula (I), (I′) and/or (I″) may be purified, for instance by reversed-phase chromatography or passage through ion exchange resins, such as for instance a strongly acidic Amberlite® resin.

All the steps above described for preparing compounds of the invention can be finished off, if necessary and if desired, with for instance evaporation, filtration, solvent extraction, distillation, chromatography, crystallization, or other operations according to the methods known to the person skilled in the art.

In the context of the present invention, the expression “hydrotropic ether or glycol” refers in particular to a highly polar solvent belonging to the class of hydroxy-alkyl ethers or alkylene glycol hydroxy-alkyl ethers which is characterized by high volatility and allows to increase the solubility of hydrophobic organic molecules having low solubility or being insoluble in water or in an aqueous phase of compositions comprising them. Such solvents are miscible in water in all proportions and can facilitate the formation of transparent and homogeneous solutions. In some cases, particularly in the presence of lyophilic compounds forming an oil phase, they can eventually provide colloidally dispersed systems.

The terms “C₁-C₆ alkyl” or “C₃-C₁₀ alkyl” refer to saturated hydrocarbon groups containing from 1 to 6 or from 3 to 10 carbon atoms respectively, which may be linear or branched. Preferably, the alkyl groups are selected from methyl, ethyl, propyl, isopropyl, butyl, isobutyl, hexyl, isohexyl, heptyl and isoheptyl.

The term “C₁-C₆ alkyl-carbonyl” refers to a carbonyl group substituted with a linear or branched saturated hydrocarbon group containing from 1 to 6 carbon atoms. Preferably the methyl-carbonyl (acetyl) group is used.

The expression “low percentage of water” refers to an amount of water lower than or equal to 15% v/v with respect to the amount of hydrotropic solvent. For instance, the amount of water can be comprised between 0.1% to 15% v/v, also considering small percentages of water already included as crystallization water in the hydrotropic (not anhydrous) solvents. In these cases, no further water is added in the reactor and the process can substantially proceed without the addition of further water.

The present invention also encompasses stereoisomers, hydrates, solvates, organic or mineral salts of the compounds of the invention, preferably of compounds with formula (I). Their tautomeric, enantiomeric, diastereoisomeric and epimeric forms are also encompassed.

In one embodiment, the invention relates to the use of a mixture comprising a solvent of the general formula (IV) as defined above in the presence of an amount of water ranging from 0.1% to 15% v/v as hydrotropic solvent in the preparation of a compound of formula (I) according to the process defined above.

In a preferred embodiment, the compound of formula (IV) is a solvent selected from 1-methoxy-2-propanol (PGME), 1-ethoxy-2-propanol (PGEE), 1-propoxy-2-propanol (PGPE), 1-butoxy-2-propanol (PnB), 3-methoxy-1-butanol (MeBuOH), propylene glycol butyl ether (PGBE), di(propylene glycol) butyl ether (DPnB), di(propylene glycol) propyl ether (DPnP), 1,3-diethoxy-2-propanol and 1-methoxy-3-butoxy-2-propanol.

More preferably the compound of formula (IV) is selected from 1-methoxy-2-propanol (PGME), 1-ethoxy-2-propanol (PGEE), 1-propoxy-2-propanol (PGPE) and propylene glycol butyl ether (PGBE). Even more preferably, the compound of formula (IV) is 1-methoxy-2-propanol (PGME).

The above hydrotropic solvents are not reprotoxic, thus the use of a mixture of a solvent of formula (IV) with a low percentage of water, as defined above, is extremely safer with respect to the use of dipolar aprotic solvents, like DMAC, for performing the reaction of the invention.

Preferably, the amount of water is comprised between 5% and 15% v/v. More preferably, it is comprised between 5% and 10% v/v.

In another embodiment of the invention, the amount of water is comprised between 0.1% and 2% v/v. In some embodiments such amount of water can be represented by the crystallization or hydration water already included in the hydrotropic solvent.

Preferably, the reaction of the invention is carried out in a mixture comprising 1-methoxy-2-propanol and an amount of water comprised between 0.1% and 2% v/v or, even more preferably, between 0.1% and 0.5% v/v, so that it can be considered that the reaction in 1-methoxy-2-propanol can be substantially carried out without addition of further water to the mixture.

In fact, it has been surprisingly found that the presence of a low percentage (equal or lower than 15% v/v) of water is helpful in solubilizing the amine of formula (III) and allows to accelerate the reaction, maintaining the homogeneity of the reaction medium while reducing its viscosity. Conversely, with amounts of water higher than 15% v/v such advantages are counteracted by the formation of several undesirable by-products, for instance formed in hydrolysis side reactions.

In another preferred embodiment, the reaction of step a) is performed at a temperature below or equal to 20° C. In fact, the control of the temperature, for instance by means of an external bath, has been found beneficial to avoid possible side-reaction related to the competitive esterification of the compound of formula (II) with the solvent of formula (IV), in particular when primary alcohols are used, and to prevent any thermal decomposition of the compounds.

A further aspect of the present invention relates to the use of a compound of formula (I), obtained as described above, for the preparation of a non-ionic X-ray contrast agent.

A preferred embodiment of the present invention relates to a process for preparing the compounds of formula (Ia) or (Ib):

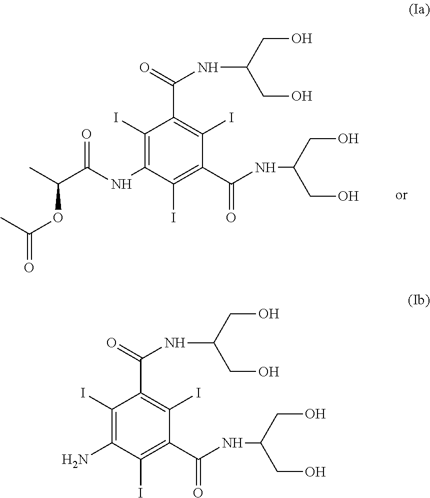 [SMILES: CC(=O)O[C@@H](C)C(=O)Nc1c(I)c(C(=O)NC(CO)CO)c(I)c(C(=O)NC(CO)CO)c1I ; "or" ; Nc1c(I)c(C(=O)NC(CO)CO)c(I)c(C(=O)NC(CO)CO)c1I]

by reacting, respectively, the compound of formula (IIa) or (IIb)

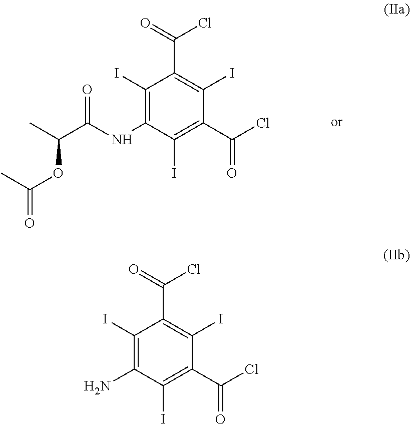 [SMILES: CC(=O)O[C@@H](C)C(=O)Nc1c(I)c(C(=O)Cl)c(I)c(C(=O)Cl)c1I ; "or" ; Nc1c(I)c(C(=O)Cl)c(I)c(C(=O)Cl)c1I]

with 2-amino-1,3-propanediol, wherein the reaction is carried out in a mixture of a compound of formula (IV), as defined above, and an amount of water ranging from 0.1% to 15% v/v.

In more preferred embodiment said reaction is carried out in a mixture of 1-methoxy-2-propanol in the presence of an amount of water ranging from 0.1% to 15% v/v. More preferably the amount of water is comprised between 0.1% and 2% v/v.

The present invention also provides for the process above described further comprising the step of
b) hydrolyzing the compound of formula (Ia) or
c) amidating the compound of formula (Ib)
   to obtain the compound of formula (V) (Iopamidol)

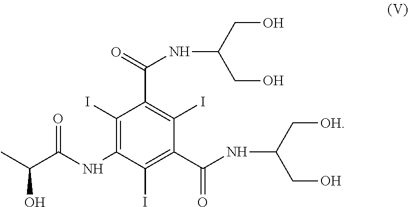 [SMILES: C[C@H](O)C(=O)Nc1c(I)c(C(=O)NC(CO)CO)c(I)c(C(=O)NC(CO)CO)c1I]

Step b) can be conveniently carried out by removing the acetyl group of compound (Ia) for instance as described in GB1472050. Step c) can be carried out by amidating the amino group of compound (Ib), for instance as described in WO2015/067601.

Another embodiment of the present invention relates to a process for preparing the compound of formula (Ic):

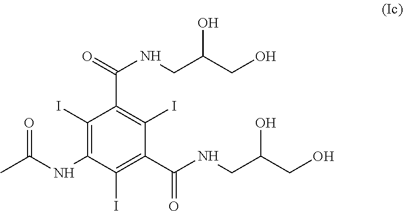 [SMILES: CC(=O)Nc1c(I)c(C(=O)NCC(O)CO)c(I)c(C(=O)NCC(O)CO)c1I]

by reacting the compound of formula (IIc)

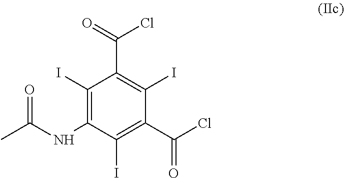 [SMILES: CC(=O)Nc1c(I)c(C(=O)Cl)c(I)c(C(=O)Cl)c1I]

with 3-amino-1,2-propanediol, wherein the reaction is carried out in a mixture of a compound of formula (IV), as defined above, and an amount of water ranging from 0.1% to 15% v/v.

In another embodiment, the invention also provides for the process above described further comprising the step of alkylating the acetyl-amino group of compound (Ic) to obtain for instance the compounds of formula (VI) (Iohexol), (VII) (Iodixanol) or (VIII) (Iopentol):

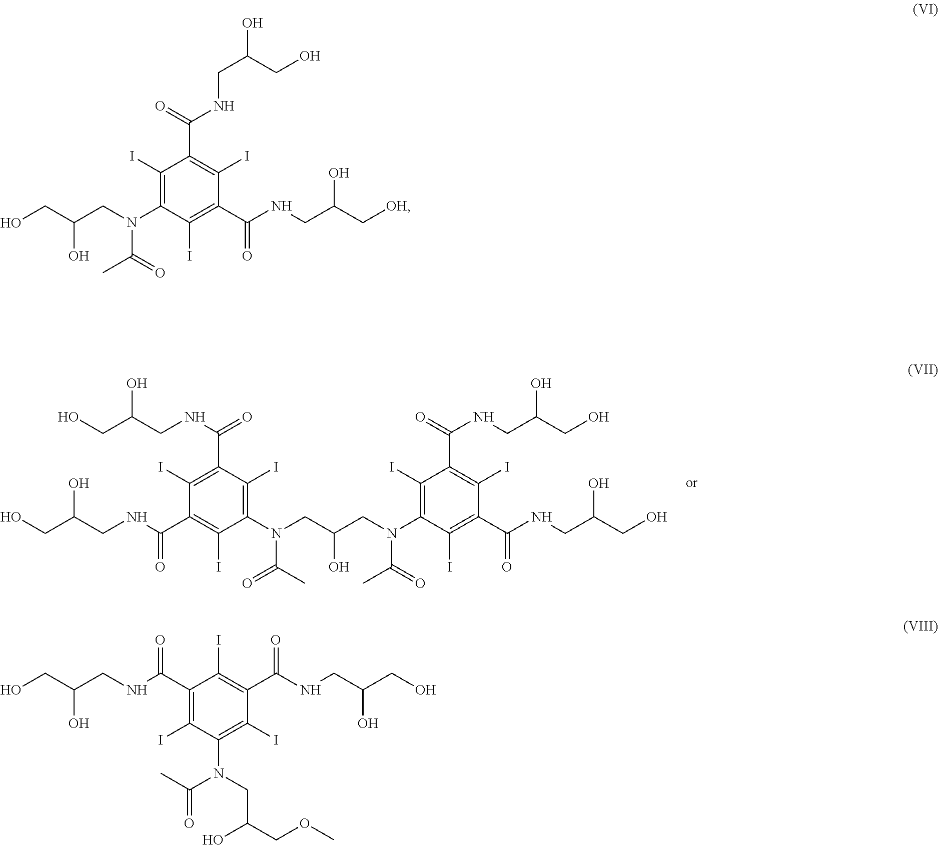 [SMILES: CC(=O)N(CC(O)CO)c1c(I)c(C(=O)NCC(O)CO)c(I)c(C(=O)NCC(O)CO)c1I ; CC(=O)N(CC(O)CN(C(C)=O)c1c(I)c(C(=O)NCC(O)CO)c(I)c(C(=O)NCC(O)CO)c1I)c1c(I)c(C(=O)NCC(O)CO)c(I)c(C(=O)NCC(O)CO)c1I ; COCC(O)CN(C(C)=O)c1c(I)c(C(=O)NCC(O)CO)c(I)c(C(=O)NCC(O)CO)c1I]

Such alkylation steps can be accomplished for instance as described respectively in U.S. Pat. No. 5,705,692 A, in ACS Omega 2018, 3, 7344-7349 and in Org. Process Res & Dev 2001, 5, 472-478.

A further embodiment of the present invention relates to a process for preparing the Intermediate of formula (Id):

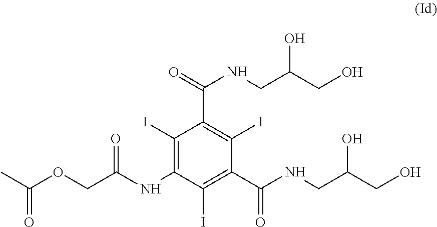 [SMILES: CC(=O)OCC(=O)Nc1c(I)c(C(=O)NCC(O)CO)c(I)c(C(=O)NCC(O)CO)c1I]

by reacting the compound of formula (IId)

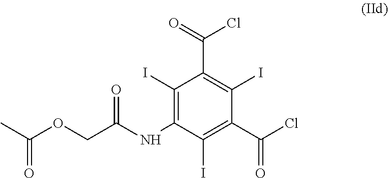 [SMILES: CC(=O)OCC(=O)Nc1c(I)c(C(=O)Cl)c(I)c(C(=O)Cl)c1I]

with 3-amino-1,2-propanediol, wherein the reaction is carried out in a mixture of a compound of formula (IV), as defined above, and an amount of water ranging from 0.1% to 15% v/v.

In another embodiment, the invention provides the process as defined above further comprising the steps of alkylating the carbonylamino group of (Id) and removing the acetyl protecting group, thus obtaining for instance the compounds of formula (IX) (Ioversol) or (X) (Iomeprol):

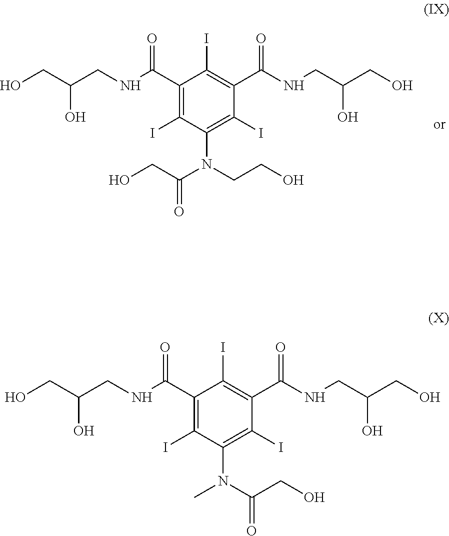 [SMILES: O=C(NCC(O)CO)c1c(I)c(C(=O)NCC(O)CO)c(I)c(N(CCO)C(=O)CO)c1I ; CN(C(=O)CO)c1c(I)c(C(=O)NCC(O)CO)c(I)c(C(=O)NCC(O)CO)c1I]

Such reactions can be carried out for instance as described respectively in U.S. Pat. No. 4,396,598 A and in U.S. Pat. No. 4,352,788 A.

A further embodiment of the present invention relates to a process for preparing the intermediate of formula (Ie):

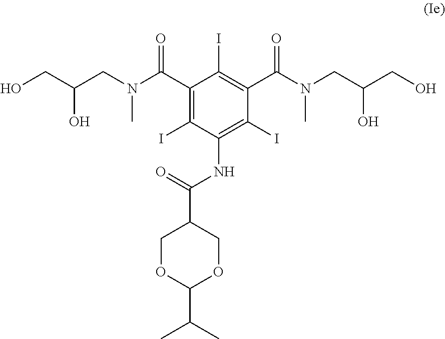 [SMILES: CC(C)C1OCC(C(=O)Nc2c(I)c(C(=O)N(C)CC(O)CO)c(I)c(C(=O)N(C)CC(O)CO)c2I)CO1]

by reacting the compound of formula (IIe)

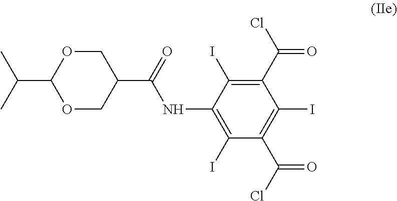 [SMILES: CC(C)C1OCC(C(=O)Nc2c(I)c(C(=O)Cl)c(I)c(C(=O)Cl)c2I)CO1]

with 3-(methylamino) propane-1,2-diol, wherein the reaction is carried out in a mixture of a compound of formula (IV), as defined above, and an amount of water ranging from 0.1% to 15% v/v.

In another embodiment, the invention provides the process as defined above further comprising the steps of removing the protecting group from compound (Ie) to obtain the compound of formula (XI) (Iobitridol)

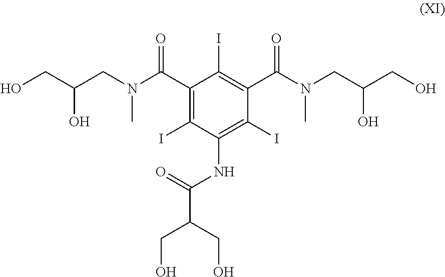 [SMILES: CN(CC(O)CO)C(=O)c1c(I)c(NC(=O)C(CO)CO)c(I)c(C(=O)N(C)CC(O)CO)c1I]

This step can be carried out for instance as described in U.S. Pat. No. 5,043,152 A.

According to the invention, the amidation reaction of step a) may be carried out in a flask or a reactor with mechanical stirring and is preferably maintained under a predetermined temperature, for instance using a cooling bath or a jacketed reactor. For example, the reaction can be performed at temperature below or equal to 20° C., preferably at a temperature of between 10° C. and 20° C., even more preferably at a temperature of between 15° C. and 18° C. In fact, it has been surprisingly found that with said ranges of temperature it is possible to keep under control any eventual thermal decomposition of reactants (e.g. esterification of the acyl chloride of formula (II) by the solvent) and formation of impurities due to the exothermic effect of the amidation reaction, although the solubility of serinol can be partially reduced. In this case, the presence of a percentage of water comprised between 0.1% v/v and 15% v/v was found also helpful to solubilize serinol in the reaction mixture at temperatures below or equal to 20° C.

Depending on the solubility of the starting materials and reagents in the reaction medium, the order of addition of the components can be modulated in different ways to foster the homogeneity and cleanliness of the mixture. Generally, the compound of formula (II) is added portion wise in solid form to a solution of the amine (III) in the mixture of compound (IV) and 0.1-15% v/v of water. Alternatively, a solution of the amine (III) dissolved in water can be added dropwise to a solution (or suspension) of compound (II) in the solvent of formula (IV). Dropwise addition can last for a period of from 0.5 min to 2 h, while the temperature is kept constant, for instance below 20° C.

Preferably, the concentration of the compound of formula (II) dissolved in the reaction medium is comprised between 0.25 M and 0.75 M. More preferably it is comprised between 0.4 M and 0.6 M.

Generally, a molar excess of the amine of formula (III) is added to the reaction. The molar ratio between the amine of formula (III) and the acyl chloride of formula (II) is comprised between 3:1 and 7:1. This range was found advantageous to reduce as much as possible the amount of the amine of formula (III) and the associated costs in case of scale-up to an industrial plant without compromising the purity of the product. In fact, it was found that, in the absence of a base or a consistent excess of amine of formula (III), several undesired by-products could be formed due to the reaction of esterification of the compound of formula (II) undertaken by the solvent competing with the amidation reaction.

Preferably, the molar ratio between the amine of formula (III) and the acyl chloride of formula (II) is comprised between 4:1 and 6:1, more preferably it is 4.5:1 or 5:1.

The reaction time may range from 3 to 20 hours to reach complete conversion. Preferably it ranges from 4 to 6 hours.

At the end of the reaction all volatile components are evaporated and the mixture is dissolved in water and purified via elution through a Amberlite IRC-120H column, followed by evaporation of the water.

If necessary, the product may be purified, for instance by preparative chromatography. More preferably the product obtained in step a), after removal of the solvent by evaporation, is directly used in step b) by addition of water, without any further purification step.

Step b) of hydrolysis of the crude obtained in step a) is generally carried out according to standard methods. Preferably the hydrolysis is carried out in a aqueous solution of NaOH, preferably with aqueous 30% NaOH. The hydrolysis step is preferably performed at room temperature for a period comprised between 1 h and 4 hours, preferably between 2 and 3 hours.

Step c) of alkylation or amidation of the intermediate obtained in steps a) or b) is generally carried out according to standard methods.

Such processes for preparing compounds of the invention can be finished off, if necessary and if desired, with evaporation, solvent extraction or distillation, filtration, chromatography or other operations according to the methods known to those skilled in the art.

The following examples illustrate the best experimental conditions to carry out the process of the invention.

### Experimental Part

All the above-mentioned compounds of formula (IV) are commercially available and were purchased from Merck KGaA, except for 1-methoxy-3-butoxy-2-propanol which was prepared as described in Leal-Duaso A. et al, Org. Process Res. Dev. 2020, 24 (2): 154-162.

In the examples below as well as throughout the application, the following abbreviations have the following meanings. If not defined, the terms have their generally accepted meanings.

List of abbreviations

| | |
|---|---|
| DMAC | N,N-dimethylacetamide |
| DMF | N,N-dimethylformamide |
| NEP | 1-ethylpyrrolidin-2-one |
| NMP | 1-methylpyrrolidin-2-one |
| PGME | 1-methoxypropan-2-ol (propylene glycol monomethyl ether) |
| PGPE | 1-propoxypropan-2-ol (propylene glycol monopropyl ether) |
| PGEE | 1-propoxypropan-2-ol (propylene glycol ethyl ether) |
| PnB | 1-butoxypropan-2-ol (propylene glycol butyl ether) |
| MeBuOH | 3-methoxybutan-1-ol |
| PGBE | 3-buthoxy-propan-1-ol (propylene glycol butyl ether) |
| DPnB | 1-[(1-butoxypropan-2-yl)oxy]propan-2-ol (di(propylene |
| | glycol) butyl ether) |
| DPnP | 1-[(1-propoxypropan-2-yl)oxy]propan-2-ol (di(propylene |
| | glycol) propyl ether) |

### Analytical Methods

The reaction mixtures and the products were analysed by High Performance Liquid Chromatography (HPLC). Such analyses were carried out on Agilent Zorbax SB-Phenyl, 5 μm, 4.6×250 mm columns using an HPLC system equipped with an UV/VIS detector.

Method 1: Mobile phase A was water and mobile phase B was acetonitrile. Gradient was hold at 0% B for 18 min, then raised from 0% to 38% B in 22 min and from 38% to 50% B in further 5 min. UV detection at 240 nm. Flow rate: 1 ml/min. Temperature: 60° C.

The yields reported below were calculated from the area percent of the HPLC chromatogram peaks.

### Example 1

### Preparation of Compound (Ia), Intermediate for the Synthesis of Iopamidol (V)

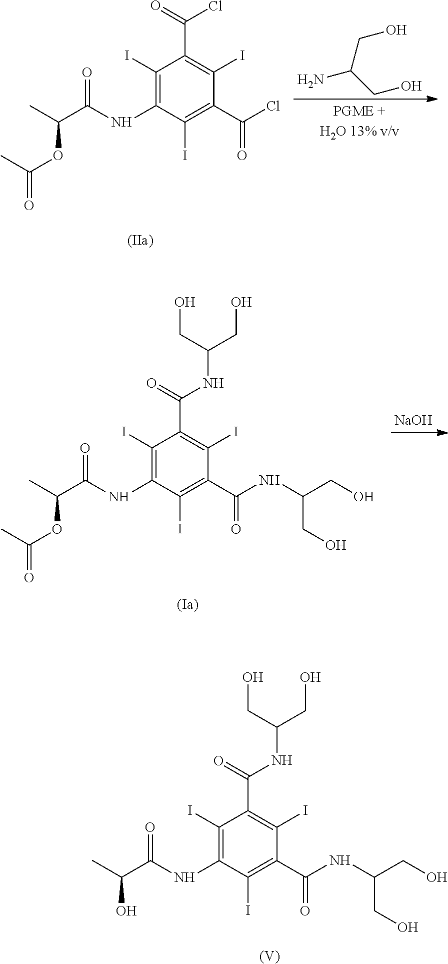 [SMILES: CC(=O)O[C@@H](C)C(=O)Nc1c(I)c(C(=O)Cl)c(I)c(C(=O)Cl)c1I ; NC(CO)CO ; CC(=O)O[C@@H](C)C(=O)Nc1c(I)c(C(=O)NC(CO)CO)c(I)c(C(=O)NC(CO)CO)c1I ; "NaOH" ; C[C@H](O)C(=O)Nc1c(I)c(C(=O)NC(CO)CO)c(I)c(C(=O)NC(CO)CO)c1I ; "(V)"]

An excess of 2-amino-1,3 propanediol (12.8 g; 141 mmol; 5 eq.) was dissolved in a mixture of PGME and water (13% v/v; 25 mL) and this solution was slowly added at 20° C. to a suspension of 5-[[2-(acetyloxy)-1-oxopropyl]amino]-2,4,6-triiodo-1,3-benzenedicarbonyl dichloride (IIa) (20 g; 28.2 mmol; 1 eq.) in PGME (30 mL). The suspension slowly turned into a clear, homogeneous brownish-yellow solution which was stirred at 20° C. for 10 h, then evaporated.

The residue of (Ia) thus obtained was dissolved with water (30 mL), then 30% aq. NaOH (11 mL) was added and the mixture stirred for 3 h at 20° C. The solution was neutralized with conc. HCl and eluted on Amberlite IR120H resin column. The eluate was evaporated to give Iopamidol (yield 93%).

The procedure described above was applied analogously using other solvents of formula (IV) of the invention or different percentages of water, obtaining the results reported in the following Table 1 in terms of final yield of iopamidol.

TABLE 1

| Compound of | Amount of water | Yield of iopamidol |
|---|---|---|
| formula (IV) | (% v/v) | (HPLC area %) |
| PGME | 5% | 94% |
| PGME | 9% | 91% |
| PGME | 15% | 86% |
| PGPE | 13% | 85% |
| PGBE | 9% | 82% |
| DPnB | 9% | 70% |
| DPnP | 9% | 79% |
| PGEE | 9% | 84% |
| MeBuOH | 9% | 84% |

### Example 2

### Preparation of Compound (Ia) in a Mixture of PGME and 0.1% v/v Water

An excess of 2-amino-1,3 propanediol (17.3 g; 190 mmol; 4.5 eq.) was dissolved at 60° C. in 57 mL of a mixture of PGME and 0.1% v/v of water, then the solution was cooled to 20° C. and slowly added in 2 hours to a suspension of S-5-[[2-(acetyloxy)-1-oxopropyl]amino]-2,4,6-triiodo-1,3-benzenedicarboxylic acid dichloride (IIa) (30 g; 42.3 mmol) at 20° C. The mixture was stirred at 20° C. for 5 hours then filtered to separate the white precipitate of 2-amino-1,3 propanediol hydrochloride formed. Aqueous 30% NaOH (30 g) was added to the filtered solution, obtaining an emulsion that was stirred for 2 hours. The solution was neutralized with HCl, filtered to remove NaCl and evaporated. The residue was dissolved with water and eluted on Amberlite IR120H resin column. The eluate was evaporated to give Iopamidol (yield 92%).

### Example 3

### Preparation of Compound (Ib)

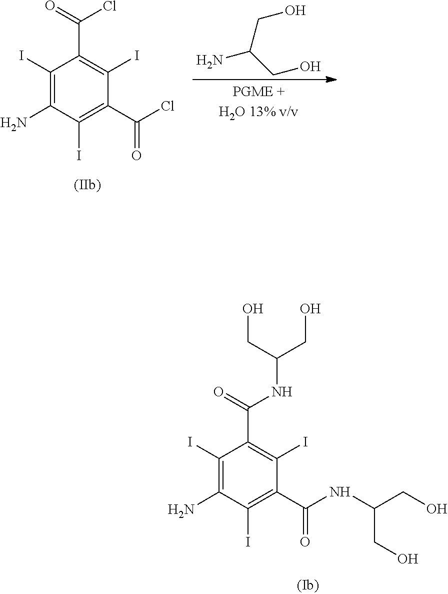 [SMILES: Nc1c(I)c(C(=O)Cl)c(I)c(C(=O)Cl)c1I ; NC(CO)CO ; Nc1c(I)c(C(=O)NC(CO)CO)c(I)c(C(=O)NC(CO)CO)c1I]

In a jacketed reactor (T=18° C.), 5-amino-2,4,6-triiodo-1,3-benzenedicarbonyl dichloride (10.00 g, 16.73 mmol) was dispersed in PGME (22 mL) under magnetic stirring. 2-amino-1,3 propanediol (6.865, 75.29 mmol) was dissolved in a solution of PGME (11 mL) and water (0.33 mL), while heating on a plate at 60° C. The solution was added dropwise in the reaction mixture in 60 minutes. By the end of the addition the mixture turned into a solution and the precipitation of a grey solid corresponding to 2-amino-1,3 propanediol chlorohydrate was then observed. The reaction was monitored via TLC (BuOH, AcOH, H₂O, 7:2:1). After 5 hours the conversion was complete. The raw mixture was filtered on fritted disc funnel and washed with 10 mL of PGME. The solvent was removed under reduced pressure and the solid obtained was then sonicated with 75 ml of water and ground with a rotor-stator homogenizer. After filtration on Buchner funnel the product was obtained as white solid (9.76 g, mass recovery: 83%, HPLC area %: 92%).

### Example 4

### Preparation of Compound (Ic)

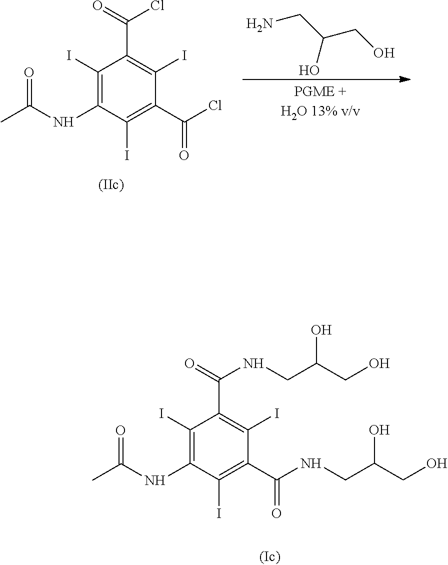 [SMILES: CC(=O)Nc1c(I)c(C(=O)Cl)c(I)c(C(=O)Cl)c1I ; NCC(O)CO ; CC(=O)Nc1c(I)c(C(=O)NCC(O)CO)c(I)c(C(=O)NCC(O)CO)c1I]

5-(acetyl-amino)-2,4,6-triiodo-1,3-benzenedicarbonyl dichloride (30.00 g, 47.04 mmol) was suspended in 26 mL of PGME in a jacketed reactor mechanically stirred. A solution of 3-amino-1,2-propanediol (18.86 g, 207 mmol) in 60 mL of PGME was added dropwise during 2 hours. The mixture was mechanically stirred for 24 hours. 200 ml of water were added and the suspension was filtered on a fritted disc funnel. The white solid obtained was washed with 50 ml of water and dried in vacuum at 50° C. overnight (35.10 g, mass recovery: 79%, HPLC area %: 96%).

### Example 5

### Preparation of Compound (Id)

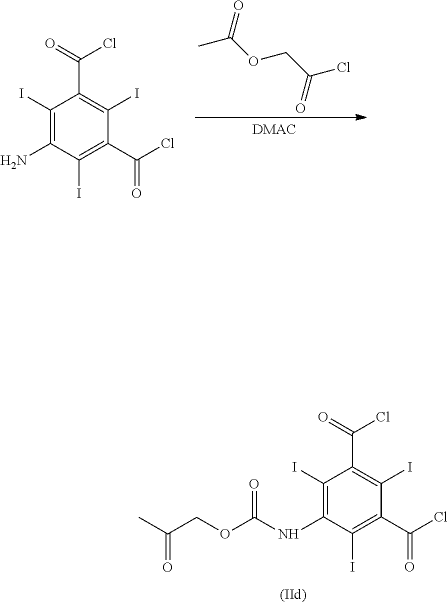

5-amino-2,4,6-triiodo-1,3-benzenedicarbonyl dichloride (5.957 g, 10.00 mmol) was dispersed in 6.7 mL of DMAC in a round bottom flask, magnetically stirred, equipped with a CaCl₂ valve and cooled at 0° C. A solution of acetoxyacetyl chloride (2.090 g, 15.30 mmol) in 13.5 mL of DMAC was added dropwise in reaction flask. After 2 hours the cooling bath was removed and the mixture was stirred at room temperature overnight. The mixture was poured into 80 mL of deionized water, a grey solid was formed which was filtered and washed with 10 ml of water. The product (6.857 g, 98.55%) was dried in vacuum at 40° C. overnight.

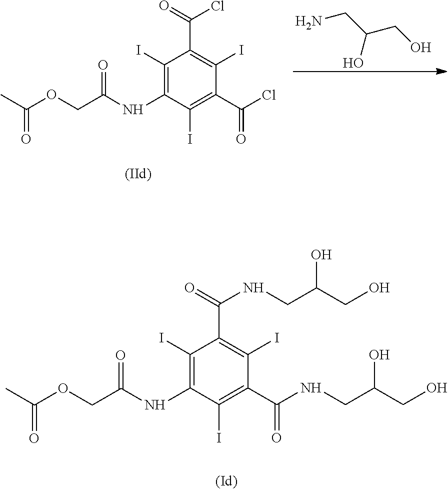 [SMILES: CC(=O)OCC(=O)Nc1c(I)c(C(=O)Cl)c(I)c(C(=O)Cl)c1I ; NCC(O)CO ; CC(=O)OCC(=O)Nc1c(I)c(C(=O)NCC(O)CO)c(I)c(C(=O)NCC(O)CO)c1I]

The acyl chloride (13.91 g, 19.99 mmol) was dispersed in 12.5 mL of PGME in a double neck, round bottom flask, via mechanical stirring. The flask was maintained at 18° C. with a cooling bath. During 60 minutes, a solution of 8.010 g of 3-amino-1,2-propanediol in 26 mL of PGME was poured into the reaction flask. The reaction, monitored via TLC, was completed in 18 hours.

### REFERENCES

1. Lusic, H. et al, Chem. Rev. 2013, 113, 1641-1666
2. Shi M. et al., Org. Process Res. Dev 2020, 24:1543-1548
3. EP 1075462
4. CN104098484
5. Subbarao C. V. et al, Chem. Eng. Technol. 2012, 35:225-237
6. Dhapte V. et al, St. Petersburg Polytechnical University Journal: Physics and Mathematics 2015, 1:424-435
7. GB1472050
8. WO2015/067601
9. U.S. Pat. No. 5,705,692 A
10. ACS Omega 2018, 3, 7344-7349
11. Org. Process Res & Dev 2001, 5, 472-478
12. U.S. Pat. No. 4,396,598 A
13. U.S. Pat. No. 4,352,788 A
14. U.S. Pat. No. 5,043,152 A
15. Leal-Duaso A. et al, Org. Process Res. Dev. 2020, 24 (2): 154-162

1. A process for the preparation of a 2,4,6-triiodoisophthalic bisamide of formula (I) 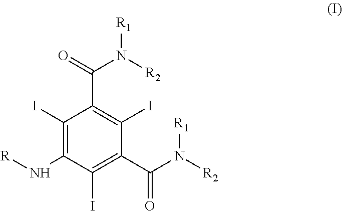
wherein
R is selected from hydrogen, a linear or branched C₁-C₆ alkyl-carbonyl, optionally substituted by —OCOCH₃, and a group 2-(propan-2-yl)-1,3-dioxane-5-carbonyl;
R₁ is each independently hydrogen or methyl; and
R₂ is each independently a group —CH(CH₂OH)₂ or —CH₂CH(OH)CH₂OH,
said process comprising the step of
a) reacting a substituted 2,4,6-triiodo-1,3-benzenedicarboxylic acid dichloride of formula (II) 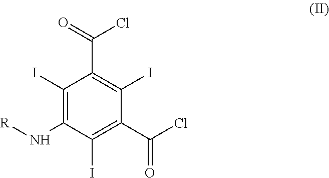
wherein R is as defined above,
with an amine of formula (III)
R₁—NH—R₂ (III)
wherein R₁ and R₂ are as defined above,
characterized in that the reaction of step a) is carried out in a mixture comprising a solvent selected from hydrotropic ethers and glycols in the presence of an amount of water ranging from 0.1% and 15% v/v and in that the molar ratio between the amine of formula (III) and the acyl chloride of formula (II) is comprised between 3:1 and 7:1.

2. The process according to claim 1 wherein said solvent is a compound of formula (IV)
R₃—O—R₄—OH (IV)
wherein
R₃ is a linear or branched C₁-C₆ alkyl, and
R₄ is a linear or branched C₃-C₁₀ alkyl, optionally interrupted by at least one oxygen atom.

3. The process according to claim 1 wherein R is hydrogen or a group selected from:

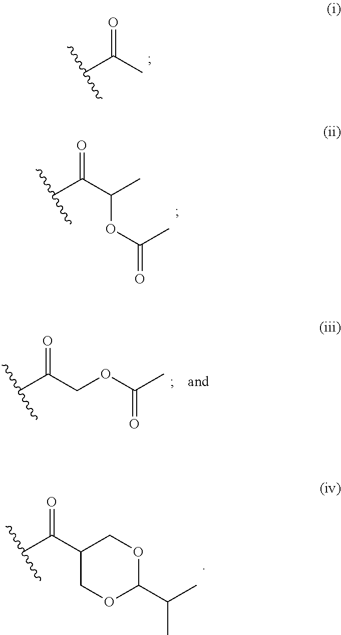

4. The process according to claim 3, wherein R is a group CH₃CO— (i).

5. The process according to claim 2, wherein the compound of formula (IV) is a solvent selected from 1-methoxy-2-propanol, 1-ethoxy-2-propanol, 1-propoxy-2-propanol, 1-butoxy-2-propanol, 3-methoxy-1-butanol, propylene glycol butyl ether, di(propylene glycol) butyl ether, di(propylene glycol) propyl ether, 1,3-diethoxy-2-propanol and 1-methoxy-3-butoxy-2-propanol.

6. The process according to claim 5, wherein the compound of formula (IV) is 1-methoxy-2-propanol.

7. The process according to claim 1, wherein the amount of water in the mixture of step a) is comprised between 5% and 15% v/v.

8. The process according to claim 1, wherein the mixture of step a) comprises 1-methoxy-2-propanol and an amount of water comprised between 0.1% and 2% v/v.

9. The process according to claim 1, wherein the amine of formula (III) is an aminoalcohol selected from 2-amino-1,3-propanediol, 3-amino-1,2-propanediol and 3-(methylamino) propane-1,2-diol.

10. The process according to claim 1 wherein the step a) is carried out at a temperature comprised between 10° C. and 20° C.

11. The process according to claim 10 wherein the step a) is carried out at a temperature comprised between 15° C. and 18° C.

12. The process according to claim 1 wherein the concentration of the compound of formula (II) is comprised between 0.25 M and 0.75 M.

13. The process according to claim 1 wherein the molar ratio between the amine of formula (III) and the acyl chloride of formula (II) is comprised between 4:1 and 6:1.

14. The process according to claim 1 further comprising the following steps:
b) hydrolyzing the compound of formula (I) and/or removing any protecting group to obtain the corresponding compound of formula (I′) 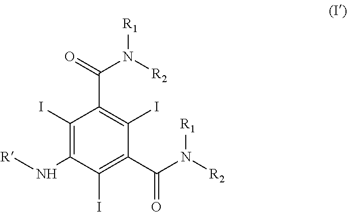
wherein R₁ and R₂ are as defined above and R′ is selected from a linear or branched C₁-C₆ alkyl-carbonyl, optionally substituted by one or more groups-OH; and/or
c) alkylating or amidating the compound of formula (I) or (I′) to obtain a compound of formula (I″) 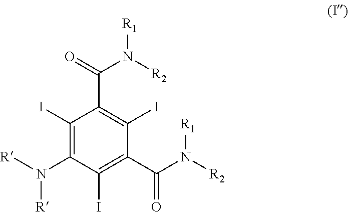
wherein R₁, R₂ and R′ are as defined above and R″ is a linear or branched C₁-C₆ alkyl or C₁-C₆ alkyl-carbonyl, optionally substituted by one or more groups —OH and/or —OCH₃.

15. (canceled)
